(19) United States
(12) Patent Application Publication
(10) Pub. No.: US 2005/0203506 A1
(43) Pub. Date: Sep. 15, 2005

(54) Protective device for electrosurgical probes

(76) Inventors: Frank Luemkemann, Jena OT Drackendorf (DE); Dirk Preuss, Jena (DE)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 11/076,502

(22) Filed: Mar. 9, 2005

(30) Foreign Application Priority Data
Mar. 9, 2004 (DE) 10 2004 011 301.7

(51) Int. Cl. A61B 18/14

(52) U.S. Cl. 606/41

(57) ABSTRACT

The invention is directed to a protective device for electrosurgical probes which are provided particularly in ophthalmology for intraocular treatment of the eye and comprise two electrodes that are arranged coaxial to one another, an insulator being arranged therebetween, and the two electrodes make contact with a connection of a current and voltage device. In order to provide a protective device for the inner electrode that can be built so as to operate in a simple manner, can be produced economically and ensures sufficient safety for operation in surgical procedures, it is proposed that an electrosurgical probe has, in its front portion, a protective device in which an inner electrode and an insulator are held in a protected manner in a reference position by an outer electrode which is movable in the longitudinal axis of the electrosurgical probe. A user releases an electrode tip of the inner electrode in a working position by initiating an axial movement process of the outer electrode. The electrosurgical probe has a handle element for handling the electrosurgical probe and the protective device.

### CROSS-REFERENCE TO RELATED APPLICATIONS

This application claims priority of German Application No. 10 2004 011 301.7, filed Mar. 9, 2004, the complete disclosure of which is hereby incorporated by reference.

### BACKGROUND OF THE INVENTION

a) Field of the Invention

The invention is directed to a protective device for electrosurgical probes which are provided particularly in ophthalmology for intraocular treatment of the eye and comprise two electrodes that are arranged coaxial to one another, an insulator being arranged therebetween, and the two electrodes make contact with a connection of a current and voltage device.
b) Description of the Related Art

Electrosurgical probes which are commonly used in ophthalmology as bipolar intraocular probes are employed particularly as electrical diathermy probes in intraocular treatment of the eye by introducing the probes through an artificially produced opening in the eye. The probes are also applied, for example, for retinal incisions through the use of a cutting instrument in the form of a plasma knife. In this case, the probe is again brought to the operating area by way of an artificially produced opening in the eye. In known embodiment forms, most electrosurgical probes work by generating an electrical field between the electrodes that are arranged coaxial to one another in that the inner electrode is constructed as an electrically conductive, axially insulated electrode, particularly formed by wire. It is important for the operation of the probes that the inner electrode projects out on the side of the probe facing the opening beyond the insulator surrounding the inner electrode. The projecting end of the inner electrode is left bare for a high-voltage discharge in order to generate a microplasma. The outer electrode is fashioned as a bare or partially insulated electrode. The insulator surrounding the inner electrode can also be used to conduct light as is suggested in DE 101 18 464. When a probe of the type mentioned above is used in ophthalmic surgery, it must be stable and easy to handle. Successful use of these probes depends upon safely carrying out extremely small incisions in the eye and minimizing the risk of injury to the patient during the operation. Due to the small wire diameter and the fact that the opening in the eye frequently closes due to inherent tensions of the tissue, the wire end of the inner electrode can bend when inserting the tip of the electrode into the opening. This severely impairs the handling and cutting efficiency of the probes, causing difficulty for the user of the probe. In addition, this presents a considerable risk of injury to the patient.

### OBJECT AND SUMMARY OF THE INVENTION

It is the primary object of the invention to provide a stable and easily handled probe for microsurgery with a protective device for the inner electrode which can be built so as to operate in a simple manner, can be produced economically and ensures sufficient safety for operation in surgical procedures.

According to the invention, this object is met in that an electrosurgical probe has, in its front portion, a protective device in which an inner electrode and an insulator are held in a protected manner in a reference position by an outer electrode which is movable in the longitudinal axis of the electrosurgical probe, a user releases an electrode tip of the inner electrode into a working position by initiating an axial movement process of the outer electrode, and the electrosurgical probe has a handle element for handling the probe and the protective device.

The outer electrode is preferably guided by a front portion in a holding element so as to be movable in a longitudinal axis of the electrosurgical probe and is blocked in axial direction in a positive engagement by means of a blocking element provided in the holding element until released, and an end portion of the outer electrode is fixedly connected to the handle element of the electrosurgical probe by means of an intermediate piece.

The blocking element which is arranged in the holding element transverse to the longitudinal axis of the electrosurgical probe advantageously has a recess, particularly a slit-shaped recess, through which the front portion of the outer electrode can be guided, and the movement of the front portion of the outer electrode is blocked in axial direction by a clamping piece which is fixedly arranged in front of the blocking element on the front portion of the outer electrode.

In an advantageous embodiment form, a tension spring is arranged in back of the blocking element, is fastened to the movable front portion and to the end portion of the outer electrode, and draws the clamping piece arranged in front of the blocking element toward the slit-shaped recess in the reference position of the outer electrode.

In an advantageous further development, an opening provided in the center of the blocking element has a diameter that is greater than the diameter of the clamping piece so that the front portion of the outer electrode is drawn back by the spring force and releases the inner electrode.

Finally, it is advantageous that the movement of the outer electrode can be carried out once or multiple times depending on the intraocular treatment of the eye for which the electrosurgical probe is used. For this purpose, the movement of the outer electrode is triggered by means of an actuating element provided at the blocking element.

In a preferred variant, the outer electrode is guided with the front portion in the holding element so as to be movable in the longitudinal axis of the electrosurgical probe and the movement of the outer electrode is blocked in axial direction until release by a force acting radially on the outer surface of the outer electrode by means of a frictionally locking holding mechanism.

In another advantageous variant of a protective device for electrosurgical probes for special areas of use, the inner electrode is mechanically protected in the reference position by a biodegradable substance which dissolves in the operating field in the work position in the eye and can be rinsed away by means of a rinsing solution. The substance is composed in such a way that when it dissolves no substrate residues remain which could impair the cutting efficiency and cutting function.

It is essential to the new electrosurgical probe with a protective device that its safe use in ophthalmic surgery is considerably improved for the user and the possible risk of injury to the patient is substantially reduced, particularly when the wire diameter of the inner electrode is very small and when problems can arise due to tensions occurring at the insertion site in the tissue causing the opening to close. This is prevented in that the outer electrode is movable in a holding element of the protective device arranged in the probe and the movement of the outer electrode is blocked in a reference position by means of a positive-locking or friction-locking holding mechanism until the electrosurgical probe is in the working position at the eye and the outer electrode which is drawn back by spring force releases the inner electrode with its bare end in that the blocking is canceled. Another substantial advantage consists in that this protective device is constructed in a simple manner and is easy to handle and can be used for different electrosurgical probes in variations corresponding to its use. For special surgical procedures, the inner electrode can also be protected when inserted into the eye by a biodegradable substance which dissolves particularly in the operating field and which can be rinsed away during the operation by means of a rinsing solution.

The invention will be described more fully in the following with reference to an embodiment example shown schematically in the drawings.

### BRIEF DESCRIPTION OF THE DRAWINGS

In the drawings:

FIG. 1 shows an embodiment example of a probe according to the invention with a protective device seen from the front in section;

FIG. 2 shows a side view of the probe in section;

FIG. 3 shows a blocking device with a positive-locking holding mechanism in section; and

FIG. 4 shows a detail of the blocking device.

### DESCRIPTION OF THE PREFERRED EMBODIMENTS

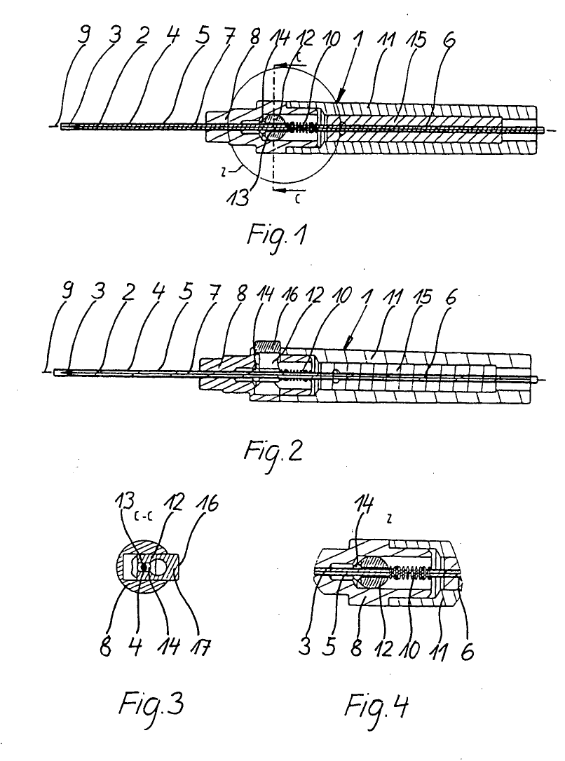

The electrosurgical probe 1 shown in section in FIGS. 1 and 2 comprises a coaxial arrangement of an inner electrode 2, particularly made from tungsten or platinum, for supplying via a high-voltage line, not shown, a high-voltage pulse needed to generate plasma. The inner electrode 2 is received by an outer electrode 4 which is constructed in particular as a high-grade or stainless steel cannula. An insulator 7 is provided between the inner electrode 2 and the outer electrode 4.

The inner electrode 2 of the electrosurgical probe 1 with an electrode tip 3 is covered in the reference position by the outer electrode 4 corresponding to FIGS. 1 to 4 when inserted into an eye of a patient in order to protect the electrode tip 3 of the inner electrode 2 from damage, e.g., breaking, when inserting into the eye. When using the electrosurgical probe 1, the electrode tip 3 of the inner electrode 2 is first released in the working position in the eye after placing the electrosurgical probe 1 on the location to be treated. For this purpose, the outer electrode 4 is guided by a front portion 5 into a holding element 8 in axial direction so as to be longitudinally movable and can be displaced in a longitudinal axis 9 of the electrosurgical probe 1. The front portion 5 of the outer electrode 4 is displaced in a defined work position by a force of a tension spring 10 acting axially in order to release the electrode tip 3 of the inner electrode 2 for insertion after placement on the operating site in the eye. The holding element 8 is fastened in a handle element 11 for handling the electrosurgical probe 1 and the protective device. For this purpose, the movement of the front potion 5 of the outer electrode 4 along the axis 9 is blocked by a blocking element 12 until release and a user can then cancel the blocking when working with the electrosurgical probe 1 by initiating a movement of the front portion 5 of the outer electrode 4 manually or automatically at any time. For this purpose, a slit-shaped recess 13 is provided in the blocking element 12, the front portion 5 of the outer electrode 4 being guided through this recess 13 (FIG. 3). A clamping piece 14 is fixedly arranged on the front portion 5 of the outer electrode 4 in front of the blocking element 12 and a tension spring 10 is fixedly connected to the front portion 5 of the outer electrode 4 on the other side of the blocking element 12. An end portion 6 of the outer electrode 4 is fixedly connected to the handle element 11 by means of an intermediate piece 15. The tension spring 10 is fastened to the stationary end portion 6 of the outer electrode 4 in such a way that the clamping piece 14 is drawn against the blocking element 12 in the reference position of the outer electrode 4 and therefore no movement of the front portion 5 of the outer electrode 4 is possible. A user who manually initiates a movement of the outer electrode 4 in the longitudinal axis 9 then moves the blocking element 12 transverse to the longitudinal axis 9 of the electrosurgical probe 1. In so doing, the blocking element 12 is guided in the holding element 8. The user initiates the movement by means of an actuating element 16. The blocking element 12 is displaced transverse to the longitudinal axis 9 of the electrosurgical probe 1 until an opening 17 of the blocking member 12 lies in the center of the longitudinal axis 9 of the electrosurgical probe 1. The diameter of the opening 17 is larger than the diameter of the clamping piece 14 so that the tension spring 10 can draw back the front portion 5 of the outer electrode 4 in this position and release the electrode tip 3 of the inner electrode 2. The movement process of the outer electrode 4 can be carried out one time for disposable electrosurgical probes 1 or multiple times for products whose residual parts are sterilized.

In another embodiment example which is not discussed in more detail, the outer electrode 4 is held with the front portion 5 in the holding element 8 in the longitudinal axis 9 of the electrosurgical probe 1 by means of a frictionally-locking holding mechanism. The movement of the outer electrode 4 in the longitudinal axis 9 of the electrosurgical probe 1 is blocked until release by a force acting radially on the outer surface of the front portion 5 of the outer electrode 4.

In another embodiment example of the electrosurgical probe 1 that is not shown in more detail, the inner electrode 2 is enclosed by a biodegradable substance that provides mechanical protection when the electrode tip 3 of the inner electrode 2 is inserted into an eye of a patient and dissolves during the operation in that it changes its aggregate state and is rinsed away with the rinsing solution used when working so that no substance residues remain which could impair operation, e.g., with respect to cutting efficiency or the cutting function itself.

While the foregoing description and drawings represent the present invention, it will be obvious to those skilled in the art that various changes may be made therein without departing from the true spirit and scope of the present invention.

Reference Numbers

| | |
|---|---|
| 1 | electrosurgical probe |
| 2 | inner electrode |
| 3 | electrode tip of the inner electrode |
| 4 | outer electrode |
| 5 | front portion of the outer electrode |
| 6 | end portion of the outer electrode |
| 7 | insulator |
| 8 | holding element |
| 9 | longitudinal axis of the probe |
| 10 | tension spring |
| 11 | handle element |
| 12 | blocking element |
| 13 | slit-shaped recess |
| 14 | clamping piece |
| 15 | intermediate piece |
| 16 | actuating element |
| 17 | opening |

1. A protective device for use in combination with electrosurgical probes which are provided particularly in ophthalmology for intraocular treatment of the eye, said electrosurgical probes comprising:
two electrodes that are arranged coaxial to one another;
an insulator being arranged therebetween; and
said two electrodes making contact with a connection of a current and voltage device;
said electrosurgical probe having, in its front portion, a protective device in which an inner electrode and an insulator are held in a protected manner in a reference position by an outer electrode which is movable in the longitudinal axis of the electrosurgical probe;
a user releasing an electrode tip of the inner electrode in a working position by initiating an axial movement process of the outer electrode; and
said electrosurgical probe having a handle element for handling the electrosurgical probe and the protective device.

2. The protective device according to claim 1, wherein the outer electrode is guided by a front portion in a holding element so as to be movable in a longitudinal axis of the electrosurgical probe and is blocked in axial direction in a positive engagement by a blocking element provided in the holding element until released, and an end portion of the outer electrode is fixedly connected to the handle element of the electrosurgical probe by an intermediate piece.

3. The protective device according to claim 1, wherein the blocking element which is arranged in the holding element transverse to the longitudinal axis of the electrosurgical probe has a recess, particularly a slit-shaped recess, through which the front portion of the outer electrode can be guided, and the movement of the front portion of the outer electrode is blocked in axial direction by a clamping piece which is fixedly arranged in front of the blocking element on the front portion of the outer electrode.

4. The protective device according to claim 1, wherein a tension spring is arranged in back of the blocking element, is fastened to the movable front portion and to the end portion of the outer electrode, and draws the clamping piece arranged in front of the blocking element toward the slit-shaped recess in the blocking element.

5. The protective device according to claim 1, wherein an opening provided in the center of the blocking element has a diameter that is greater than the diameter of the clamping piece.

6. The protective device according to claim 1, wherein the movement of the outer electrode is triggered by an actuating element provided at the blocking element.

7. The protective device according to claim 1, wherein the movement of the outer electrode can be carried out once or repeatedly.

8. The protective device according to claim 1, wherein the outer electrode is guided with the front portion in the holding element so as to be movable in the longitudinal axis of the electrosurgical probe, and the movement of the outer electrode is blocked in axial direction until release by a force acting radially on the outer surface of the outer electrode by means of a frictionally locking holding mechanism.

9. An electrosurgical probe having a protective device, said probe being provided particularly in ophthalmology for intraocular treatment of the eye and comprises:
two electrodes that are arranged coaxial to one another;
an insulator being arranged therebetween;
said two electrodes making contact with a connection of a current and voltage device; and
a protective device for the inner electrode being provided by a biodegradable substance which dissolves particularly in the operating field in the eye and can be rinsed away by a rinsing solution.
